(19) United States
(12) Patent Application Publication
(10) Pub. No.: US 2023/0357126 A1
(43) Pub. Date: Nov. 9, 2023

(54) PROCESS FOR PREPARATION OF TERT-BUTYLAMINE AND PROPIONIC ACID SALTS FROM N- TERTIARY BUTYL ACRYLAMIDE

(71) Applicant: PRACHI SPECIALTIES, Pune (IN)

(72) Inventor: Prashant BARVE, Pune (IN)

(21) Appl. No.: 18/040,261

(22) PCT Filed: Jul. 29, 2020
(86) PCT No.: PCT/IN2020/050662

§ 371 Date: Feb. 1, 2023

(30) Foreign Application Priority Data
May 4, 2020 (IN) 202021018881

(51) Int. Cl. C07C 209/62; C07C 209/84; C07C 51/41

(52) U.S. Cl. CPC C07C 209/62; C07C 209/84; C07C 51/41

(57) ABSTRACT

Disclosed is a process (100) for conversion of N-tertiary butyl acrylamide to tert-butylamine and salts of propionic acid. The process comprises of first selectively reducing the vinylic double bond in N-tertiary butyl acrylamide by catalytic hydrogenation of an alcoholic solution of N-tertiary butyl acrylamide to provide N-tertiary butyl propanamide; recovering the hydrogenation catalyst by filtering the solution and treating the solution with an alkali to produce N-tertiary butylamine and corresponding alkali salt of propionic acid. The process converts of N-tertiary butyl acrylamide into value added products at milder reaction conditions, without producing any hazardous byproducts and effluents.

### FIELD OF THE INVENTION

The present invention relates to a process for the preparation of Propionic acid salts and tert-butylamine More particularly the said process is related to preparation of highly pure Calcium/Sodium Propionate and highly pure N-tertiary butylamine from N-tertiary butyl acrylamide.

### BACKGROUND OF THE INVENTION

Tert-butylamine is a clear, colorless liquid with an ammonia-like odor and it is used as intermediate in manufacturing of active pharmaceutical ingredients. Tert-butylamine also finds applications in polymer industry, as an intermediate for rubber accelerators, insecticides, fungicides, dyestuffs and pharmaceuticals. Calcium propionate/Sodium propionate find its application in food additives and specialty/performance chemicals.

U.S. Pat. No. 3,806,543A, discloses a method of manufacture of N-tert-butylamine by cleaving any of the N-tert-alkylamides of the formula R₁—CONH—R₂, in which R₁ means a saturated or unsaturated hydrocarbon group having 2 or 3 carbon atoms or the methoxy-derivative of such hydrocarbon group, and R₂ represents a tert-alkyl group. As per the method disclosed, N-Tertiarybutyl acrylamide is converted hydrolytically to tert-butylamine and Sodium acrylate, as sown in Scheme-I below:

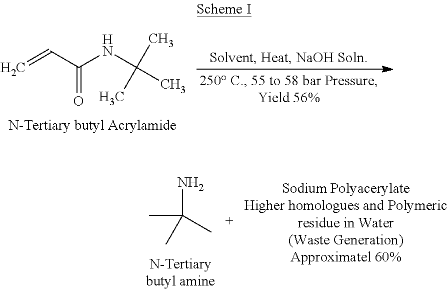

In this reaction, 1500 Kg of N-tertiary butyl acrylamide is dissolved in 1500 kg of Methanol and treated with 1040 kg of 48% Caustic lye. The reaction takes place at 250° C. and at 55 to 58 bar pressure. The reaction is carried out in a continuous stirred tank reactor (CSTR) made of Nickel. The reaction is completed in 2 to 2.5 hours, where the reaction efficiency is 86%. Tert-butylamine being low boiler is removed by distillation, whereas Sodium acrylate is obtained as residue. This process has several disadvantages as below:
The process mentioned in Scheme I is highly hazardous to practice commercially for continuous production.
The alkaline condition at very high temperature is corrosive to various metals and needs very special material of construction.
After conversion of N-tertiary butyl acrylamide to tert-butylamine, the product i.e. tert-butylamine gets sold in the market very easily, whereas the sodium acrylate byproduct, which is not of good quality, gets converted to unwanted oligomers and polymer and further it is highly soluble in water which increases the COD and BOD values of waste water to undesirable values and very difficult for effluent treatment. In short, the total molecule does not get converted into value added product.
The vinylic double bond (electrons) in the molecule (i.e. N-tertiary butyl acrylamide) as well as the lone pair on adjacent Nitrogen gets delocalized to carbonyl carbon (C═O), thus the δ+ charge on this carbon gets reduced, further to it the electron inductive effect of tertiary butyl group is also towards this carbon. This is the reason; the hydrolytic cleavage of N-tertiary butyl acrylamide to tert-butylamine is very difficult and needs very high temperature even in highly alkaline conditions.

Accordingly, there exists a need to provide a method for converting N-Tertiary butyl acrylamide to tert-butylamine, which will be less hazardous, completely green, without any side products or effluents, and which will give highly pure products.

### OBJECTS OF THE INVENTION

An object of the present invention is to provide a green process for conversion of N-tertiary butyl acrylamide to tert-butylamine and propionic acid salts.

Another object of the present invention is to provide highly pure tert-butylamine and propionic acid salts from N-tertiary butyl acrylamide.

Still another object of the present invention is to provide a process for conversion of N-tertiary butyl acrylamide to tert-butylamine and propionic acid salts that will not produce any hazardous side products or effluents.

Yet another object of the present invention is to provide a process for conversion of N-tertiary butyl acrylamide to tert-butylamine and propionic acid salts that will take place in milder reaction conditions.

### SUMMARY OF THE INVENTION

The present invention discloses a process for preparation of tert-butylamine and propionic acid salts from N-Tert-butyl acrylamide.

In first step, the reactants—N-tert-butyl acrylamide, an organic solvent, and a hydrogenation catalyst are charged into a first reactor and the reactor is flushed several times with nitrogen. In an embodiment, the organic solvent is the any one selected from methanol, ethanol, isopropyl alcohol and tertiary butyl alcohol; preferably methanol. In another embodiment, the hydrogenation catalyst is any one selected from Pd/C, Pt/C, Raney Nickel, sodium borohydride and Sodium bis(2-methoxyethoxy) aluminium hydride. The first reactor is a high pressure stirred reactor. Further, the reaction mixture in step is agitated at a speed ranging from 600 rpm to 800 rpm and heated up to a temperature ranging from 65° C. to 75° C. Hydrogen gas is introduced into the first reactor under pressure ranging from 10 kg-g/cm² to 20 kg-g/cm² while agitating and maintaining the reaction mixture at a temperature ranging from 125° C. to 145° C. Hydrogen consumption is continuously monitored and reaction continued till hydrogen consumption is stopped. At the end of the reaction, N-tert-butyl propanamide is obtained in solution form by selective reduction of vinylic double bond in N-tert-butyl acrylamide. The reaction mass is cooled to room temperature and filtered to separate and recover the hydrogenation catalyst. The filtrate obtained is solution of N-tertiary butyl propanamide in organic solvent.

Further, hydrolysis of N-tertiary butyl propanamide is carried out by transferring the solution to a second reactor fitted with heating jacket, internal cooling coil, pressure gauge, vent valve, pressure regulator, vent condenser and product receiver; and charging the second reactor with 11% to 12% w/v aqueous alkali solution. In an embodiment, the alkali is any one selected from sodium hydroxide, potassium hydroxide and lime. The reaction mass is heated to a temperature ranging from 270° C. to 275° C. under autogenous pressure ranging from of 55 kg-g/cm² to 60 kg-g/cm². The pressure is released through vent condenser and the condensate containing 18 to 19% by wt of tert-butylamine, 69 to 70% by wt of organic solvent and balance water is collected in the product receiver. The reaction mass remained in the second reactor contains alkali metal propionate in water. The condensate is distilled under reflux till the distillation still top reaches to temperature ranging from 97° C. to 98° C. and a tert-butylamine rich distillate in organic solvent is collected. The tert-butylamine rich distillate along with 48% caustic lye is charged to a fractional distillation column and distilled under reflux at a vapor temperature ranging from 39° C. to 43° C. to obtain 99% to 99.7% pure tert-butylamine. The reaction mass remained in the second reactor is evaporated in a rotary evaporator to obtain 90% to 95% pure alkali metal propionate in crystalline form.

### DETAILED DESCRIPTION OF THE EMBODIMENTS

The foregoing objects of the invention are accomplished and the problems and shortcomings associated with prior art techniques and approaches are overcome by the present invention described in the present embodiments.

Accordingly, a process of preparation of tert-butylamine and propionic acid salts from N-Tert-butyl acrylamide comprises reducing the vinylic double bond in N-tertiary butyl acrylamide is reduced to give N-tertiary butyl propanamide by catalytic hydrogenation in first step (Scheme II):

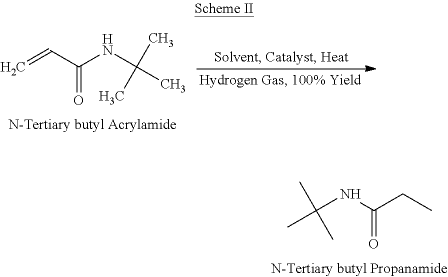

N-tertiary butyl acrylamide used in the reaction can be a crude material or wet purified material.

In next step, the above solution of product i.e. N-tertiary butyl propanamide is filtered to recover and recycle the hydrogenation catalyst. Reaction in scheme I can be carried out using either fresh hydrogenation catalyst or recycled catalyst obtained from earlier batch. The solution of product (N-tertiary butyl propanamide) is further treated with an alkali selected from NaOH, KOH and Lime (Ca(OH)₂) to produce N-tertiarybutylamine and corresponding alkali salt of Propionic acid, as shown in Scheme IIA, Scheme IIB and Scheme IIC.

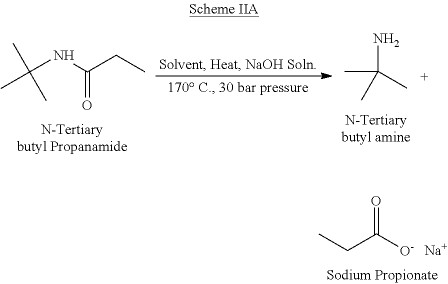

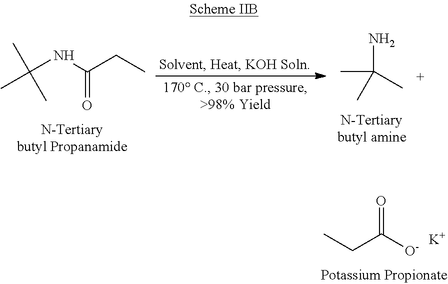

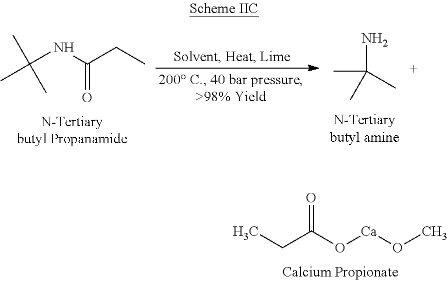

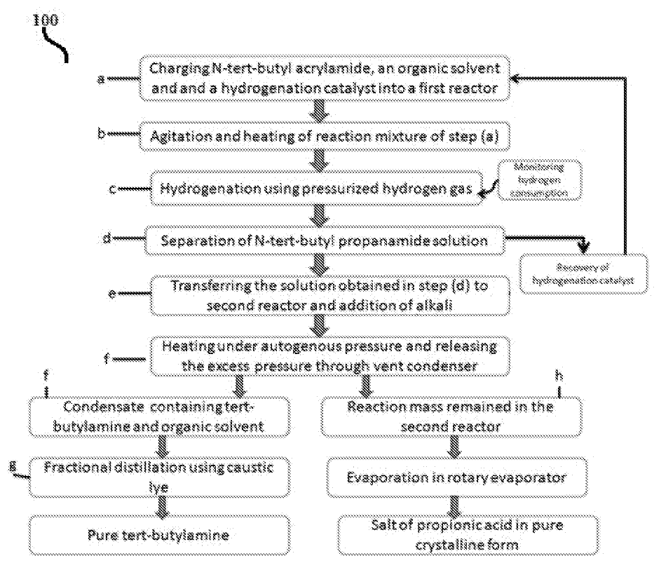

The process is illustrated with reference to the accompanying drawings. Referring to FIG. 1, a process (100) for preparation of tert-butylamine and propionic acid salts from N-Tert-butyl acrylamide is shown.

In step (a), the process (100) consists of charging N-tert-butyl acrylamide (30% to 36% w/v); an organic solvent (69% to 63% v/v); and a hydrogenation catalyst (1% w/v) into a first reactor and flushing the reactor several times with nitrogen, wherein the first reactor is a high pressure stirred reactor. In an embodiment, the solvent is any one selected from methyl alcohol, ethyl alcohol, isopropyl alcohol, tertiary butyl alcohol and in a preferred embodiment, the solvent is methyl alcohol. In another embodiment, the hydrogenation catalyst is any one selected from Pd/C, Pt/C, RaNi, Sodium boro-hydride and Sodium bis (2-methoxyethoxy) aluminium hydride i.e. vitride.

In step (b), the reaction mixture in step (a) is heated up to a temperature ranging from 65° C. to 75° C., while agitating at a speed ranging from 600 rpm to 800 rpm.

In step (c), hydrogen gas under pressure ranging from 10 kg-g/cm² to 20 kg-g/cm² is introduced into the first reactor and the reaction mixture is further heated and maintained at a temperature ranging from 125° C. to 145° C., while agitating the mixture and continuously monitoring the hydrogen consumption. Monitoring of hydrogen consumption is continued for a time period ranging from 0.5 hrs to 5 hrs, until hydrogen consumption is stopped. At the end of this reaction, N-tert-butyl propanamide is obtained in solution form by selective reduction of vinylic double bond in N-tert-butyl acrylamide.

In step (d), the solution obtained in step (c) is cooled to room temperature. The cooled mass is filtered to separate and recover the hydrogenation catalyst from N-tertiary butyl propanamide solution. The resulting N-tertiary butyl propanamide solution analyzed by GLC shows single peak product. This is completely green process and does not produce any side products or effluents.

In step (e) the N-tert-butyl propanamide solution obtained in step (d) is transferred into a second reactor. The second reactor is a high pressure stirred reactor fitted with heating jacket, internal cooling coil, pressure gauge, vent valve, pressure regulator, vent condenser and product receiver. 11% to 12% w/v aqueous solution of alkali selected from sodium hydroxide, potassium hydroxide and lime is charged to the second reactor and it is flushed with nitrogen gas.

In step (f), the reaction mass in step (e) is heated to a temperature ranging from 270° C. to 275° C. under autogenous pressure ranging from of 55 kg-g/cm² to 60 kg/cm². The pressure is released through vent condenser and condensed hydrolysis product is collected in the product receiver. The condensed hydrolysis product contains 18 to 19% by wt of tert-butylamine, 69 to 70% by wt of organic solvent and balance water, while the reaction mass remained in the second reactor contains alkali metal propionate in water.

In step (g), the condensed hydrolysis product obtained in step (f) tert-butylamine is separated from the organic solvent by fractional distillation and the organic solvent is recovered. The tert-butylamine rich distillate contained around 44 to 45% tert-butylamine and balance solvent (methanol). Tert-butylamine is distilled under reflux in the range of 39° C. to 43° C. vapor temperature. Tertiary butylamine being highly volatile (boiling point 42° C.) is easily separated in pure form. Fractional distillation gives 99% to 99.7% pure tert-butylamine and the recovery is 94%.

In step (h), the reaction mass obtained in step (f) is charged to a stirred reactor and pH of the solution is adjusted to 7 to 7.5 by adding small amount of propionic acid. By adding 1% activated carbon, the pH adjusted solution is refluxed for 15 minutes and filtered to remove waste carbon. The clear solution is charged to rotary evaporator and concentrated under reduced pressure, to obtain alkali metal propionate in crystalline form.

The process (100) is further explained by way of illustrative examples. The examples are given by way of illustration and should not be construed to limit the scope of present invention.

(A) Preparation of N-Tertiary Butyl Acrylamide

N-tertiary butyl acrylamide is the starting material in the process for preparation of tert-butylamine. Illustrative examples A-1, A-2, A-3 and A-4 given below explain the process of preparation of N-tertiary butyl acrylamide and the process of purification of the same.

### Example A-1: Preparation of N-Tertiary Butyl Acrylamide Using Acrylonitrile and Isobutylene Gas

In a five liter capacity all glass stirred reactor placed in a hot water bath, 1240 gm of 70% Sulfuric acid solution in water was charged. Then to this flask was charged 480 gm of acrylonitrile along with 1.74 gm of monomethyl ether of hydroquinone (MeHQ) under stirring followed by 384 gm of Isobutylene gas was injected through the reaction mass maintained at 43 to 45° C. The reaction mass was stirred for 24 hours. Then the reaction mass was transferred to ten liter capacity stirred reactor containing 2500 gm of cold water. As a result the crude product was obtained in slurry form. Then the reaction mass was filtered to isolate crude product (N-tertiary butyl acrylamide). The product cake was washed with 250 gm of water. The wet cake 1087 gm had 23.62% moisture (as titrated by KF) and had 0.47% as sulfate. The yield of product was 94.74% based on dry weight based on Isobutylene input. The mother liquor and washings were preserved for recycle as explained in next Example.

### Example A-2: Preparation of N-Tertiary Butyl Acrylamide with Recycles of Mother Liquor of Example A-1

The mother liquor obtained under Example A-1 was recovered under vacuum on the rotary evaporator till 1240 gm of concentrated mass was obtained. This concentrate was charged in a five liter capacity all glass stirred reactor placed in a hot water bath. Then to this flask was charged 480 gm of Acrylonitrile along with 1.74 gm of Monomethyl ether of Hydroquinone (MeHQ) under stirring followed by 384 gm of Isobutylene gas was injected through the reaction mass maintained at 43 to 45° C. The reaction mass was stirred for 24 hours. Then to the reaction mass was transferred to ten liter capacity stirred reactor containing 2500 gm of cold water. As a result the crude product was obtained in slurry form. Then the reaction mass was filtered to isolate crude product (N-tertiary butyl acrylamide). The product cake was washed with 250 gm of water. The wet cake 1240 gm had 33.69% moisture (as titrated by KF) and had 0.87% as sulfate. The yield of product was 93.18% based on dry weight based on Isobutylene input. The mother liquor and washings were preserved for recycle and was recycled with three more times as explained under Example-A-2, without losing the yield.

### Example A-3: Preparation of N-Tertiary Butyl Acrylamide Using Acrylonitrile and Isobutylene Gas

In a five liter capacity all glass stirred reactor placed in a hot water bath, 1150 gm of 69.93% Methane sulfonic acid solution in water was charged. Then to this flask was charged 593 gm of Acrylonitrile along with 3.78 gm of Monomethyl ether of Hydroquinone (MeHQ) under stirring followed by 467 gm of Isobutylene gas is injected through the reaction mass maintained at 49 to 50° C. The reaction mass was stirred for 8 hours. Then the reaction mass was transferred to ten liter capacity stirred reactor containing 1150 gm of cold water. As a result the crude product was obtained in slurry form. Then the reaction mass was filtered to isolate crude product (N-tertiary butyl acrylamide). The product cake was washed with 250 gm of water. The wet cake 1222 gm had 20.89% moisture (as titrated by KF) and had 2.01% as acidity. The yield of product was 88.95% based on dry weight based on Isobutylene input. The mother liquor and washings were preserved for recycle.

### Example A-4

Purification of Crude N-Tertiary Butyl Acrylamide

500 gm of methanol was taken in a stirred reactor and 500 gm of crude N-tertiary butyl acrylamide obtained from Example A-1 was transferred to it. It was converted to solution in methanol. 100 mg of Monomethyl ether of Hydroquinone (MeHQ) was added to it, followed by addition of 25 ml of 10% sodium carbonate solution in water. Then 5 gm of activated carbon was charged to it and the reaction mass was heated for 15 minutes till reflux and then filtered the spent carbon. More than 1000 ml of water was charged to the reaction mass after cooling, so as to precipitate purified N-tertiary butyl acrylamide. The wet cake of purified N-tertiary butyl acrylamide was washed with small amount of water. The wet cake of purified N-tertiary butyl acrylamide (almost 90% and above of input material) contained 21.2% moisture and no other impurity was detected after GC analysis. The filtrate was subjected to recover methanol under vacuum. Residual quantity of N-tertiary butyl acrylamide was also recovered on cooling the bottom material and thus almost quantitative recovery of N-tertiary butyl acrylamide was done. The second crop of N-tertiary butyl acrylamide was recycled in next purification batch. Both the materials i.e. Crude N-tertiary butyl acrylamide and purified wet N-tertiary butyl acrylamide material were used in next reactions i.e. hydrogenation of vinylic double bond.

(B) Preparation of N-Tertiary Butyl Propanamide

Illustrative examples B-1 to B-6 describe hydrogenation of purified and crude N-tertiary butyl-acrylamide to N-tertiary butyl propanamide. The hydrogenation of Crude N-tertiary butyl acrylamide or purified wet N-tertiary butyl acrylamide was carried out by using either fresh catalyst or recycled catalyst obtained from earlier batch. The general hydrogenation procedure was followed as written below:

Fresh Catalyst Batch
1. An appropriate amount of starting materials (crude or purified N-tertiary butyl acrylamide equivalent to 500 gm, methanol 1000 gm with different catalysts as shown in following table) were weighed, mixed together and charged in to the SS-316 high pressure stirred reactor/autoclave.
2. The autoclave was flushed several times with nitrogen. Typically it was flushed by nitrogen gas for 5 to 7 times.
3. The agitator of reactor was started and set to 700 rpm.
4. In one set of reaction condition, reaction mass heating was started, once the temperature reached to 70° C., hydrogen was taken while reaction temperature was increased to 125 to 130° C. with pressure continuously monitored and hydrogen pressure was kept between 10-12 kg-g/cm². In an another set of reaction condition, reaction mass heating was started, once the temperature reached to 70° C., hydrogen was taken while reaction temperature was increased to 140 to 145° C. with pressure continuously monitored and hydrogen pressure was kept between 18-20 kg-g/cm².
5. The reaction was carried out until hydrogen consumption was stopped. The time required for reaction was noted.
6. After completion of reaction the autoclave was cooled down to room temperature.
7. The material was removed and filtered to recover catalyst and hydrogenated product in solution form.
8. The catalyst after washing with small amount of solvent was dried and used further for recycled batch.

Recycled Catalyst Batch

Same procedure was followed as above narrated under points 1 to point 8. In addition to the used catalyst from previous batch, 0.5 g of fresh catalyst was used.

TABLE 1
Shows the results of various experiments.

| | | | Reaction | | |
|---|---|---|---|---|---|
| Example | Type of | | Temperature/Pressure | | Time |
| Number | Catalyst | Catalyst | kg-g/cm² | Yield | In hours |
| B-1 (*) | 1% Pd/C | 5.9 gm Fresh | 140 to 145° C./18-20 | 99.95% | 0.30 |
| | (Dry) | Catalyst | | | |
| B-2 (*) | 1% Pd/C | 5.9 gm Fresh | 125 to 130° C./10-12 | 99.92% | 3.50 |
| | (Dry) | Catalyst | | | |
| B-3 (*) | 1% Pt/C | 11.8 gm Fresh | 125 to 130° C./10-12 | 99.65% | 3.30 |
| | (50% | Catalyst | | | |
| | Moist) | | | | |
| B-4 (*) | 1% Pt/C | Recycle Catalyst + | 125 to 130° C./10-12 | 99.80% | 4.30 |
| | (50% | 0.5 g fresh | | | |
| | Moist) | | | | |
| B-5 (*) | 1% Pt/C | 5.9 gm Fresh | 125 to 130° C./10-12 | 99.84% | 3.30 |
| | (Dry) | Catalyst | | | |
| B-6 (**) | 1% Pd/C | 5.9 gm Fresh | 125 to 130° C./10-12 | 99.92% | 3.50 |
| | (Dry) | Catalyst | | | |
| B-7 (*) | Raney Ni | 4.45 | 125 to 130° C./10-12 | 100.00% | 3.00 |
| | (Wet) | | | | |
| B-8 (*) | Raney Ni | 2.3080 from B-7 + | 125 to 130° C./10-12 | 100.00% | 2.00 |
| | (Wet) | 0.1010 fresh | | | |
| B-9 (*) | Raney Ni | 3.1013 | 125 to 130° C./10-12 | 100.00% | 2.00 |
| | (Wet) | | | | |
| B-10 (*) | Raney Ni | 2.44 from B-9 + | 125 to 130° C./10-12 | 100.00% | 2.00 |
| | (Wet) | 0.1404 fresh | | | |

Note-1
(*) Purified N-tertiary butyl acrylamide from Example A-4 was used during hydrogenation reaction.
(**) Crude N-tertiary butyl acrylamide from Example A-3 was used during hydrogenation reaction
Note-2
The reaction mass after hydrogenation was analyzed by GLC as follows: N-tertiary butyl propanamide sample dissolved in methanol. 0.4 μL of this solution was injected in GC column of specifications DB17 Column with length of 30 meters. ID 0.32 mm. Film thickness 0.5 μm.

Column Conditions were Maintained as Follows:

Injector temperature: 250° C.

FID temperature: 250° C.

Nitrogen flow rate: 0.9 ml/min

Oven temperature: Initial 100° C., Final 240° C.,

Rate of rise: 15°/min (Hold for 10 min)

Retention Times Observed as Follows:

Methanol: 3.2 min (solvent methanol)

N-tertiary butyl propoanamide: 10.1 min (product)

N-tertiary butyl acrylamide: 10.5 min (starting material)

(C) Separation of N-Tertiary Butyl Propanamide

Examples C-1 and C-2 illustrate method of separation of N-tertiary butyl propanamide from the filtrates obtained in examples B-1 to B-6.

### Example C-1

Separation of N-Tertiary Butyl Propanamide from Filtrates Obtained in Examples B-1 to B-5

The filtrate obtained from Examples B-1 to B-5 as mentioned in Table-1 was transferred to rotary evaporator and subjected to evaporation of solvent under reduced pressure so as to obtain molten mass which on standing was solidified. This material as such was taken for further conversion to product.

### Example C-2

Separation of N-Tertiary Butyl Propanamide from Filtrates Obtained in Example B-6

The filtrate obtained from Example B-6 as mentioned in Table 1 was treated with 50 ml of 10% solution of sodium carbonate and 400 ml of water. This mixture was transferred to rotary evaporator and subjected to evaporation of solvent under reduced pressure so as to obtain bi layer material on cooling the solidified product in slurry form was filtered to separate product. This material as such was taken for further conversion to product.

(D) Hydrolysis of N-Tertiary Butyl Propanamide to Tertiary Butylamine and Salt of Propionic Acid:

Examples D-1, D-2 and D-3 illustrate the method of obtaining tertiary butylamine and salt of propionic acid using N-tertiary butyl propanamide obtained in Examples B-1 to B-6

### Example D-1: Hydrolysis of N-Tertiary Butyl Propanamide to Tertiary Butylamine and Calcium Propionate Salt

The solution of N-tertiary butyl propanamide in methanol as stated under Examples B-1 to B-5 was transferred to 5-L capacity high pressure stirred reactor. Then slurry of 175 gm of lime (pharma grade lime) in 1500 gm of water was charged to the reactor. The reactor was equipped with heating jacket, internal cooling coil, pressure gauge, vent valve and pressure regulator, vent condenser and product receiver. After initial flushing with nitrogen, the reaction mass was heated till 270° C. to 275° C. under autogenous pressure of 55 kg-g/cm² to 60 kg-g/cm², any excess pressure above this pressure was slowly released through vent condenser and the product tert-butylamine with methanol along with small amount of water was allowed to condense and was collected continuously in the receiver. The reaction was continued for five hours. Once the excess pressure generation was complete and desired quantity of product was collected, the reaction mixture was cooled, which contains mainly solution of calcium propionate in water. Which was treated to isolate pure calcium propionate in crystalline form as explained under Example E-1.

The distillate containing tert-butylamine in methanol and water was further processed to isolate pure tert-butylamine as explained under Example F-1.

### Example D-2: Hydrolysis of N-Tertiary Butyl Propanamide to TertiaryButylamine and Calcium Propionate Salt

The solution of N-tertiary butyl propanamide in methanol as stated under Examples B-1 to B-5 was transferred to 5-L capacity high pressure stirred reactor. Then slurry of 175 gm of lime (pharma grade) in 1500 gm of water was charged to the reactor equipped with heating jacket, internal cooling coil N-tertiary butyl propanamide, pressure gauge, vent valve and pressure regulator, vent condenser and product receiver. After initial flushing with nitrogen, the reaction mass was heated till 248° C. to 250° C. under autogenous pressure of 40 to 45 kg-g/cm², any excess pressure above this pressure was slowly released through vent condenser and the product tert-butylamine with methanol along with small amount of water was allowed to condense and was collected continuously in the receiver. The reaction was continued for nine and half hour. Once the excess pressure generation was complete and desired quantity of product was collected, the reaction mixture was cooled, which contains mainly solution of calcium propionate in water. Which was treated to isolate pure calcium propionate in crystalline form as explained under Example E-1. The distillate containing tert-butylamine in methanol and water was further processed to isolate pure tert-butylamine as explained under Example F-1.

### Example D-3: Hydrolysis of N-Tertiary Butyl Propanamide to Tertiary Butylamine and Sodium Propionate Salt

The solution of N-tertiary butyl propanamide in methanol as stated under Examples B-1 to B-5 was transferred to 5-L capacity high pressure stirred reactor. Then 350 gm of 48% Caustic lye in 1250 gm of water was charged to the reactor equipped with heating jacket, internal cooling coil, pressure gauge, vent valve and pressure regulator, vent condenser and product receiver. After initial flushing with nitrogen, the reaction mass was heated till 173° C. to 175° C. under autogenous pressure of 10 to kg-g/cm², any excess pressure above this pressure was slowly released through vent condenser and the product tert-butylamine with methanol along with small amount of water was allowed to condense and was collected continuously in the receiver. The reaction was continued for nine and half hour. Once the excess pressure generation was complete and desired quantity of product was collected, the reaction mixture containing mainly solution of sodium propionate in water was cooled. Which was treated to isolate pure sodium propionate in crystalline form as explained under Example F-2. The distillate which contains tert-butylamine in methanol and water was further processed to isolate pure tert-butylamine as explained under Example F-1.

(E) Recovery of Pure Tertiary-Butyl-Amine

Example E-1 illustrates the method of recovery of pure tert-butylamine from the distillates obtained from the Example D-1, D-2 and D-3.

### Example E-1: Recovery of Pure Tertiary-Butyl-Amine

The distillate obtained from the Example D-1, D-2 and D-3 was charged to the fractional distillation still having distillation column, condenser, receiver and reflux line. The distillate contained around 18 to 19% by wt % tertiary-butyl-amine, 69 to 70% by wt % methanol and balance water. The material was distilled under atmospheric pressure under reflux till the distillation still top temperature reached to 64 to 66° C., till that time tert-butylamine rich cut in methanol was collected, the quantity collected was 620 gm. Then the distillation was continued to recover methanol as distillate till the distillation still bottom temperature reached to 97° C. to 98° C. The distillate collected in this temperature range was chiefly methanol.

The tert-butylamine rich distillate contained around 44 to 45% tert-butylamine and balance methanol. This distillate was charged to the fractional distillation still having distillation column, condenser, receiver and reflux line. Then to this mixture 1250 gm of 48% caustic lye was charged. The tert-butylamine was distilled under reflux in the range of 39° C. to 43° C. vapor temperature. The distilled tert-butylamine had a purity of 99.6% by GLC and the recovery was 94%.

(F) Recovery of Alkyl Propionate Salt

Examples F-1 and F-2 illustrate the method of recovery of propionic acid salts from the bottom material remained in the reactor.

### Example F-1

Recovery of Pure Calcium Propionate

The bottom material obtained from the Example D-1 and D-2 was charged to the stirred reactor and 1% activated carbon was charged to it. The material was refluxed for 15 minutes and was filtered to remove waste carbon. The clear solution was charged to rotary evaporator and water was concentrated out under reduced pressure till the crystals of calcium propionate started appearing in the evaporation flask. At this point the concentration was stopped and material was cooled. The calcium propionate white crystals were filtered out, dried and analyzed by HPLC.

### Example F-2

Recovery of Pure Sodium Propionate

The bottom material obtained from the Example D-3 was charged to the stirred reactor, and the reaction mass pH was adjusted to 7 to 7.5 by adding small amount of propionic acid. Then to the neutral mass 1% activated carbon was charged, the material was refluxed for 15 minutes and was filtered to remove waste carbon. The clear solution was charged to rotary evaporator and water was concentrated out under reduced pressure till the crystals of sodium propionate started appearing in the evaporation flask. At this point the concentration was stopped and material was cooled. The sodium propionate white crystals were filtered out, dried and analyzed by HPLC.

### Advantages of the Invention

1. The by-product in Acrylamido tertiary butyl sulfonic acid production is converted to four to five value added products (depending upon Scheme-IIA, IIB or IIC).
2. The main product Acrylamido tertiary butyl sulfonic acid becomes more viable due to reduction in cost of effluent treatment and the by-product generates more revenue.
3. Due to reduction of vinylic double bond, the hydrolytic cleavage of N-tertiarybutylproponamide takes place at milder conditions. Hence this cleavage is no longer highly hazardous reaction to be practiced industrially.
4. Both steps employed in this invention are green in nature, quantitative in nature and does not leave effluent.
5. Both the products i.e. tertiarybutylamine is used in the manufacturing of more than several API manufacturing. Also it is used in two applications in plastic industry. Also it has one application in cosmetic industry. Thus tertiary butylamine is an important industrial chemical.
6. Also, the other product of this reaction i.e. Alkali metal salt of Propionic acid is used as food additive, in bakery product as preservative etc. Thus it is also an industrially important chemical.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the scope of the claims of the present invention.

1. A process for preparation of tert-butylamine and propionic acid salts from N-Tert-butyl acrylamide, the process comprising steps of:
a) charging N-tert-butyl acrylamide (30% to 36% w/v); an organic solvent (69% to 63% v/v); and a hydrogenation catalyst (1% w/v) into a first reactor and flushing the reactor several times with nitrogen, wherein the first reactor is a high pressure stirred reactor;
b) agitating and heating the reaction mixture in step (a) at a speed ranging from 600 rpm to 800 rpm and up to a temperature ranging from 65° C. to 75° C.;
c) introducing hydrogen gas under pressure ranging from 10 kg-g/cm² to 20 kg/cm², into the first reactor and maintaining the reaction mixture at a temperature ranging from 125° C. to 145° C. while agitating the reaction mixture and continuously monitoring the hydrogen consumption, for a time period ranging from 0.5 hrs to 5 hrs, until hydrogen consumption is stopped, wherein N-tert-butyl propanamide is obtained in solution form by selective reduction of vinylic double bond in N-tert-butyl acrylamide;
d) cooling the reaction mass obtained in step (c) to room temperature and filtering the cooled reaction mass to separate and recover the hydrogenation catalyst from N-tertiary butyl propanamide solution;
e) transferring the N-tert-butyl propanamide solution obtained in step (d) into a second reactor, charging the second reactor with 11% to 12% w/v aqueous solution of any alkali selected from sodium hydroxide, potassium hydroxide and lime, and flushing the second reactor with nitrogen, wherein the second reactor is a high pressure stirred reactor fitted with heating jacket, internal cooling coil, pressure gauge, vent valve, pressure regulator, vent condenser and product receiver;
f) heating the reaction mass in step (e) to a temperature ranging from 270° C. to 275° C. under autogenous pressure ranging from of 55 kg-g/cm² to 60 kg-g/cm², releasing the pressure through vent condenser and collecting the condensate in the product receiver wherein the condensate contains 18 to 19% by wt of tert-butylamine, 69 to 70% by wt of organic solvent and balance water; and the reaction mass remained in the second reactor contains alkali metal propionate in water;
g) distilling the condensate obtained in step (f) under atmospheric pressure under reflux till the distillation still top reaches to temperature ranging from 97° C. to 98° C. to collect a tert-butylamine rich distillate in organic solvent; charging the tert-butylamine rich distillate in organic solvent along with 48% caustic lye to a fractional distillation column and distilling under reflux at a vapor temperature ranging from 39° C. to 43° C. to obtain tert-butylamine in pure form; and
h) concentrating the reaction mass obtained in step (f) in a rotary evaporator under reduced pressure, to obtain alkali metal propionate in crystalline form.

2. The process as claimed in claim 1, wherein the organic solvent is any one selected from methanol, ethanol, isopropyl alcohol and tertiary butyl alcohol

3. The process as claimed in claim 1, wherein the hydrogenation catalyst is any one selected from Pd/C, Pt/C, Raney Nickel, sodium borohydride and Sodium bis(2-methoxyethoxy) aluminium hydride.

4. The process as claimed in claim 1, wherein the hydrogenation catalyst is optionally the recovered hydrogenation catalyst in step (d).

5. The process as claimed in claim 1, wherein N-tertiary butyl propanamide is obtained in solid form by subjecting the filtrate obtained in step (d) to evaporation of solvent under reduced pressure.

6. The process as claimed in claim 1, wherein the product tert-butylamine is having 99.7% to 99.9% purity.

7. The process as claimed in claim 1, wherein the product propionic acid salts is having 90 to 95% purity.
